(19) United States
(12) Patent Application Publication
(10) Pub. No.: US 2025/0167027 A1
(43) Pub. Date: May 22, 2025

(54) CLOSED LOOP CONTROL SYSTEM TO MONITOR INSIDE PARAMETERS OF A SUBSTRATE CARRIER

(71) Applicant: Applied Materials, Inc., Santa Clara, CA (US)

(72) Inventors: Srinivas Poshatrahalli Gopalakrishna, Bangalore (IN); Shivaraj Nara Manjunath, Bangalore (IN); Devendra Channappa Holeyannavar, Bangalore (IN); Paul Reuter, Austin, TX (US); Douglas Baumgarten, Round Rock, TX (US); Sushant Koshti, Sunnyvale, CA (US); Amit Kumar Biswas, Bangalore (IN); Nithiyanantham Balasubramaniam, Leander, TX (US); Latha Ramesh, Bangalore (IN); Navya Talluri, Bangalore (IN)

(21) Appl. No.: 18/517,791

(22) Filed: Nov. 22, 2023

(51) Int. Cl. H01L 21/673; G01N 19/10; G01N 33/00; G06F 30/27; H01L 21/67

(52) U.S. Cl. CPC H01L 21/67389; G01N 19/10; G01N 33/0062; G06F 30/27; H01L 21/67242

(57) ABSTRACT

A method for monitoring inside parameters of a substrate carrier. The method includes receiving a first substrate carrier, supplying a fluid, at a first flow rate, through an inlet of the first substrate carrier, and at least partially purging the fluid through an outlet of the first substrate carrier for a first period of time. The method further includes measuring, using a first sensor disposed at the outlet, a first value of a first property of an exhaust from the substrate carrier at the outlet of the first substrate carrier, and determining, based at least in part on the first value of the first property, a second value of the first property inside the first substrate carrier.

### FIELD

The present disclosure relates to systems and methods for monitoring an inside environment of a substrate carrier used for storing and transporting a substrate.

### BACKGROUND

Processing substrates in semiconductor electronic device manufacturing is generally carried out in multiple process tools, where substrates travel between process tools in substrate carriers, such as, e.g., front opening unified pods (FOUPs). A FOUP may be docked at a load port of an equipment front end module (EFEM), sometimes referred to as a factory interface (FI), where one or more substrates may be transferred to a load lock, a transfer chamber and/or a process chamber. Pre- and post-substrate exposure to moisture and oxygen can cause on substrate corrosion (e.g., etch), interlayer defects (e.g., film stress and resistivity, physical vapor deposition) and device non-uniformity (e.g., chemical vapor deposition passivation). Eliminating moisture and oxygen from the EFEM environment reduces and/or eliminates such device performance and yield challenges.

### SUMMARY

The following is a simplified summary of the disclosure in order to provide a basic understanding of some aspects of the disclosure. This summary is not an extensive overview of the disclosure. It is intended to neither identify key or critical elements of the disclosure, nor delineate any scope of the particular implementations of the disclosure or any scope of the claims. Its sole purpose is to present some concepts of the disclosure in a simplified form as a prelude to the more detailed description that is presented later.

One embodiment is a method for monitoring inside parameters of a substrate carrier. The method includes receiving a first substrate carrier, supplying a fluid, at a first flow rate, through an inlet of the first substrate carrier, and at least partially purging the fluid through an outlet of the first substrate carrier for a first period of time. The method further includes measuring, using a first sensor disposed at the outlet, a first value of a first property of an exhaust from the substrate carrier at the outlet of the first substrate carrier, and determining, based at least in part on the first value of the first property, a second value of the first property inside the first substrate carrier.

Another embodiment is a system for monitoring inside parameters of a substrate carrier. The system includes a first front opening unified pod (FOUP) having an inlet to receive a fluid at a first flow rate and an outlet to at least partially purge the fluid for a first period of time. The system further includes a first sensor disposed at the outlet. The first sensor is to measure a first relative humidity (RH) or a first amount of a first gas at the outlet of the first FOUP. The system further includes a controller operatively coupled to the first sensor. The controller is configured to determine, based at least in part on the first RH or the first amount of the first gas at the outlet, a second RH or a second amount of the first gas inside the first FOUP.

Another embodiment is method including supplying a fluid, at the first flow rate, through an inlet of a substrate carrier. The method further includes at least partially purging the fluid through an outlet of the substrate carrier for a first period of time. The method further includes determining, by a first sensor disposed inside the substrate carrier, a value of a first property inside the substrate carrier. The method further includes determining, by a second sensor disposed at the outlet of the substrate carrier, a second value of the first property at the outlet of the substrate carrier. The method further includes determining a relationship between the first value of the first property and the second value of the first property. The method further includes updating a model based on the relationship between the first value of the first property and the second value of the first property, wherein the model is trained to process a measured value of the first property at an outlet of a new substrate carrier to determine an estimated value of the first property within the new substrate carrier.

### BRIEF DESCRIPTION OF THE DRAWINGS

The drawings, described below, are for illustrative purposes and are not necessarily drawn to scale. The drawings are not intended to limit the scope of the disclosure in any way.

FIG. 1A illustrates a block diagram of an example electronic device processing system according to one or more embodiments of the disclosure.

FIG. 1B illustrates a schematic view of an example system according to one or more embodiments of the disclosure.

FIG. 2 illustrates a schematic view of a first front opening unified pod (FOUP) according to one or more embodiments of the disclosure.

FIGS. 3A-3D are charts comparing relative humidity (RH) measured inside the FOUP versus RH measured at the purge outlet or exhaust port over a period of time.

FIG. 4 illustrates a flowchart of a method for monitoring inside parameters of a substrate carrier according to one or more embodiments of the disclosure.

FIG. 5 illustrates a flowchart of a method for monitoring inside parameters of a substrate carrier according to one or more embodiments of the disclosure.

FIG. 6 is a block diagram illustrating an exemplary system architecture, according to one or more embodiments of the disclosure.

FIG. 7 is a block diagram illustrating a computer system, according to one or more embodiments of the disclosure.

### DETAILED DESCRIPTION

Reference will now be made in detail to the example embodiments of this disclosure, which are illustrated in the accompanying drawings. Wherever possible, the same reference numbers will be used throughout the drawings to refer to the same or like parts throughout the several views. Features of the various embodiments described herein may be combined with each other, unless specifically noted otherwise.

EFEMs provide a non-reactive environment for transferring substrates between substrate carriers (e.g., FOUPs) and a load lock and/or chamber. This is achieved by sealing the interior volume of the EFEM as much as is practical and optionally flooding the interior volume with a gas such as nitrogen that is generally non-reactive with substrate materials. The non-reactive gas forces out any reactive gases such as oxygen from the EFEM and also reduces/eliminates moisture from the EFEM. One or more load ports for docking one or more substrate carriers may be arranged along a front face of the EFEM. The load ports of conventional EFEMs may be purged (e.g., bottom purged) with nitrogen gas (N₂) to reduce the relative humidity (RH) and/or oxygen levels within the FOUP and EFEM. However, when FOUPs are being transported, relative humidity (RH) and oxygen levels within the FOUP may increase over time and may affect device performance and result in yield challenges. For example, a FOUP may not be airtight, and over time oxygen and/or moisture from an external environment may leak into a FOUP.

In some embodiments, a method for monitoring an inside environment of a substrate carrier (e.g., a FOUP) is described. The method includes receiving a FOUP (e.g., from another location within the cleanroom or another manufacturing facility) and analyzing the inside environment of the FOUP to ensure a contamination free environment. For example, the analysis may include determining the amount of relative humidity (RH) inside the FOUP. In some embodiments, the analysis may include determining the amount of a gas (e.g., oxygen) inside the FOUP. In some embodiments, the analysis may include determining the amount of total volatile organic compounds (TVOC) inside the FOUP. In some embodiments, the analysis may include determining the number of particles (e.g., aerosol particles) inside the FOUP. In some embodiments, the analysis may include determining the temperature inside the FOUP. In some embodiments, the analysis may include determining the amount of two or more of the above parameters inside the FOUP. In embodiments, one or more of the RH, oxygen level, temperature, amount of TVOCs, amount of aerosol particles, etc. inside of a FOUP may be estimated without including any sensors within the FOUP. Instead, one or more sensors may be disposed at an outlet of the FOUP (e.g., in an exhaust line that connects to an outlet of a FOUP that is placed at a FOUP receiving area of an EFEM). Values of one or more properties of an exhaust from the FOUP may be measured using the one or more sensors, and these value(s) may be processed using a model that outputs estimates of values of the one or more properties within the FOUP. Alternatively, the analysis may be performed by installing one or more sensors (e.g., microelectromechanical system (MEMS) sensors), and measuring one or more parameters inside the FOUP. The one or more sensors may include a RH sensor, a gas sensor, a temperature sensor, a chemical sensor, or combinations thereof. In some embodiments, the FOUP may have one or more RH sensors and one or more gas sensors. Similarly, one or more sensors may be installed outside the FOUP to measure the same parameter outside the FOUP. For example, the one or more sensors may be installed in an outlet of the FOUP where a gas may be purged. The one or more sensors may be connected in series or in parallel to measure the one or more parameters.

Embodiments enable values of one or more properties within a FOUP to be estimated or measured prior to opening a door of a FOUP to combine a FOUP environment with an EFEM environment. If the estimated or measured properties of the FOUP environment fail to meet one or more criteria, then opening of the FOUP door may be delayed or prevented to reduce an exposure of an internal environment of the EFEM to the environment of the FOUP. Purging of the FOUP may continue until one or more criteria are satisfied (e.g., until a FOUP RH is at or below a RH threshold). Once the one or more criteria are satisfied, then the FOUP door may be opened into an EFEM.

Advantages of the embodiments include efficient monitoring of parameters inside the FOUP in real-time and predicting its health without opening the FOUP door as it arrives or leaves a semiconductor tool. Determining the inside environment of the substrate carrier without opening the door is advantageous because opening the door may allow contaminants to enter the substrate carrier and may impact the substrate(s) stored inside the substrate carriers. This may also result in an entire batch of substrates being discarded as a result of contamination. Other advantages include avoiding wafer scrapping and improving productivity, retrofittable modular design, and backward compatibility. Other advantages include better control and tracking of FOUP conditions and predicting FOUP health. The disclosed system may also optimize N₂ purge flow in the load ports using measured values from the sensors. The measured values can also be used to track a status of a FOUP between the process tools. The embodiments disclosed herein can be used in EFEM systems, FOUP stackers, and/or overhead hoist transport (OHT) conveyers.

When a FOUP is received (e.g., from another location within the cleanroom or another manufacturing facility), one or more parameters relating to the inside environment of the FOUP may be measured using the one or more sensors inside the FOUP. For example, an initial RH value may be measured, an initial oxygen level may be measured, an initial TVOC content may be measured, an initial temperature, and/or an initial particle (e.g., aerosol) count may be measured, and time elapsed since the last time the front door of the FOUP was closed (e.g., age) may be recorded. Since the RH and/or other parameters inside the FOUP change over time, age is recorded for comparing FOUPs with same/different ages. A database (e.g., a metadata table) may be generated including the different values for RH, oxygen, TVOC, temperature, and particle count over different ages.

In some embodiments, a test gas may be supplied to the FOUP at a first flow rate. The test gas (e.g., nitrogen (N₂) or clean dry air (CDA)) may be supplied through one or more inlets provided in the FOUP. After supplying the test gas for a period of time at the first flow rate, the test gas may be at least partially purged through one or more outlets of the FOUP. Alternatively, the test gas may be purged as the test gas is being supplied to the FOUP at the first flow rate. As the test gas is being purged from the FOUP, the one or more sensors on the outside of the FOUP measure the one or more parameters (e.g., RH, oxygen, TVOC, temperature, and/or particle count) in real-time. The outside measurements for each of the parameters may be plotted against the inside measurements for the same parameters over a period to determine a relationship between the outside measurement and the inside measured of the parameter. For example, the relationship between the outside measurement and the inside measurement may be expressed as an equation having a gradient, a y-intercept, and/or other factors. The relationship between the outside measurement and the inside measurement may vary based on the initial reading of the parameter (e.g., RH, oxygen, TVOC, temperature, and/or particle count) and the age (e.g., time elapsed since the last time the front door of the FOUP was closed) of the FOUP. In some embodiments, the initial values for the parameters, the real-times values, and the age of the FOUP may be recorded in the database, and historical values (e.g., recorded over a period of time for different FOUPs) may be used to train a machine learning model, which may then output an inside measurement based on the outside measurement and age of the FOUP. In embodiments, the model (e.g., trained ML model or equation) may be used along with measurements of a FOUP exhaust to estimate values of one or more properties of a FOUP environment.

The method may further include receiving a front opening unified pod (FOUP) at a process interface (e.g., etching, deposition, lithography, or other tools). The interface may be used to lock the FOUP in place. The FOUP may include one or more inlets to allow a fluid (e.g., nitrogen) to flow into the FOUP and one or more outlets to allow the fluid to flow out of the FOUP. After the FOUP is locked in place at the process interface, a fluid (e.g., a test gas such as nitrogen) is supplied through one or more inlets at a first flow rate. After supplying the test gas for a period of time at the first flow rate, the test gas may be at least partially purged or exhausted through one or more outlets of the FOUP. Alternatively, the test gas may be purged as the test gas is being supplied to the FOUP at the first flow rate. As the test gas is being purged from the FOUP, the one or more sensors on the outside of the FOUP (e.g., in an exhaust line connected to an outlet of the FOUP) measure the one or more parameters (e.g., RH, oxygen, TVOC, temperature, and/or particle count) in real-time. The outside measurements may be input into the model, and the model may output an estimated inside measurement based on the outside measurement and/or age (e.g., time elapsed since the last time the front door was closed) of the FOUP. If the derived value of the inside measurement is greater than a threshold value, then an alert may be generated at a load port unit (LPU) of a processing chamber including the FOUP, and the FOUP may be further treated (e.g., with an inert gas) to remove any residual RH inside the FOUP. However, if the derived value of the inside measurement is within an acceptable range (e.g., equal to or lesser than a threshold value), then the FOUP may be loaded onto the LPU and/or a FOUP door may be opened. Other embodiments relate to substrate processing systems incorporating the above-described methods.

Some embodiments relate to an independent sensing module that may be used with any bottom purge capable load port, any N₂ FOUP type, or in any stacker used in a semiconductor fabrication process. The sensing module may include a sensor (e.g., MEMS sensor) that can be disposed at the outlet of the FOUP. The sensor may determine a first relative humidity (RH) or a first property at the outlet of the FOUP. The sensing module may further include a controller operatively coupled to the sensor. The controller may be configured to determine, based at least in part on the first RH or the first property at the outlet, a second RH or a second property inside the FOUP. Any bottom purge capable load port, any N₂ FOUP type, or any stacker can be retrofit with this sensing module to measure and control a property (e.g., RH, temperature, gas content, particle count, TVOC, etc.) inside the FOUP without opening the door of the FOUP.

Embodiments are discussed with regards to FOUPs and EFEMs. However, it should be understood that the embodiments discussed with reference to FOUPs equally apply to any type of substrate carrier. Additionally, the embodiments discussed with reference to EFEMs also apply to any chamber that can receive a FOUP or other type of substrate carrier. Accordingly, it should be understood that any discussion of FOUPS applies to all types of substrate carriers and any discussion of EFEMs applies to all types of chambers that can receive substrate carriers such as FOUPs.

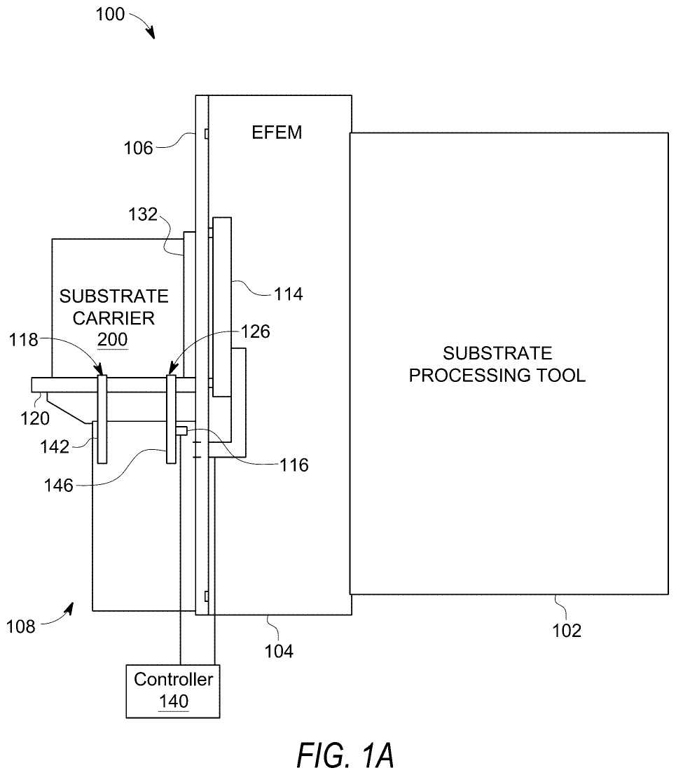

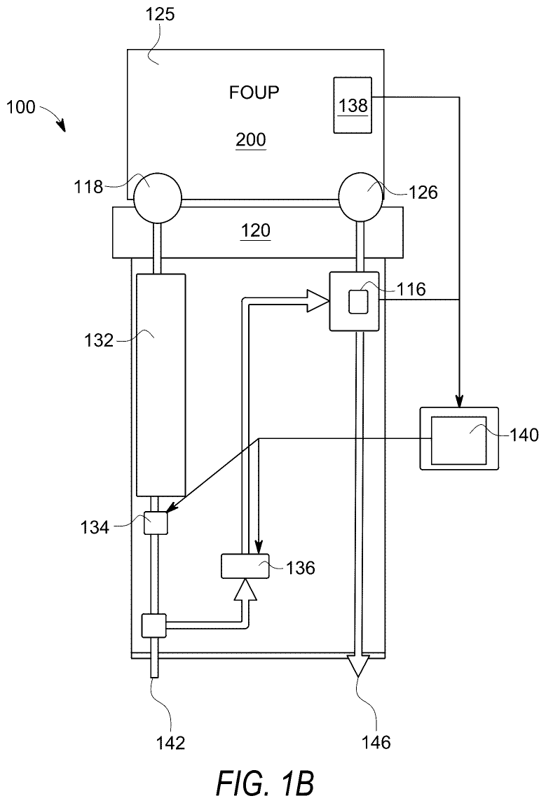

Turning to FIG. 1A, a block diagram of an example electronic device processing system 100 according to some embodiments is shown. The system 100 includes one or more substrate processing tools 102 coupled to an EFEM 104. The substrate processing tool(s) 102 may be or include a transfer chamber, one or more process chambers, one or more load lock chambers, or a combination thereof. In some embodiments, the EFEM 104 is coupled to a transfer chamber via one or more load locks. The transfer chamber may in turn be connected to one or multiple process chambers (e.g., etch chambers, deposition chambers, etc.). The EFEM 104 may be coupled to a backplane 106 of a load port 108. A docking station 120 of the load port 108 is adapted to support a substrate carrier 200 (e.g., a FOUP) which can be opened by a door opener 114 of the load port 108.

In embodiments, substrate carrier 200 includes a door 132 that separates an environment of the substrate carrier 200 from an environment of EFEM 104. The load port 108 may include an input line 142 that provides a fluid (e.g., one or more purge gases such as N₂) into substrate carrier 200 through an inlet 118 of substrate carrier 200 and an exhaust line 146 that receives an exhaust from substrate carrier 200 via outlet 126 of substrate carrier 200.

One or more sensors 116 may be coupled to exhaust line 146 of the load port 108. The one or more sensors may include, for example, a humidity sensor, an oxygen sensor, a temperature sensor, a TVOC sensor, and/or particle count sensor. The one or more sensors 116 and/or door opener 114 may be coupled to a controller 140 (also referred to as a system controller).

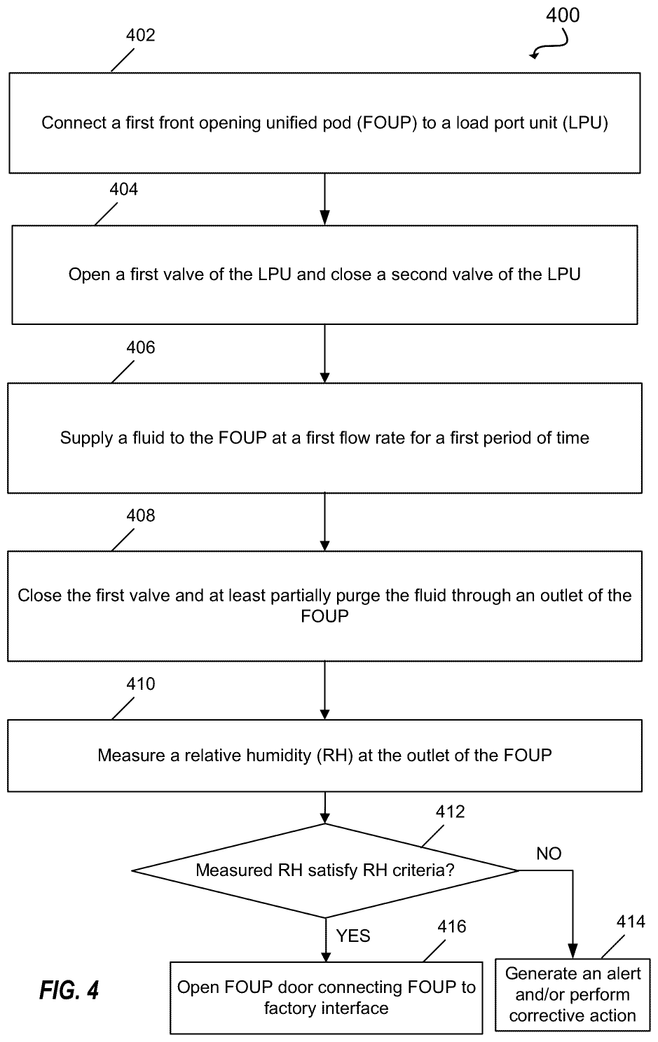

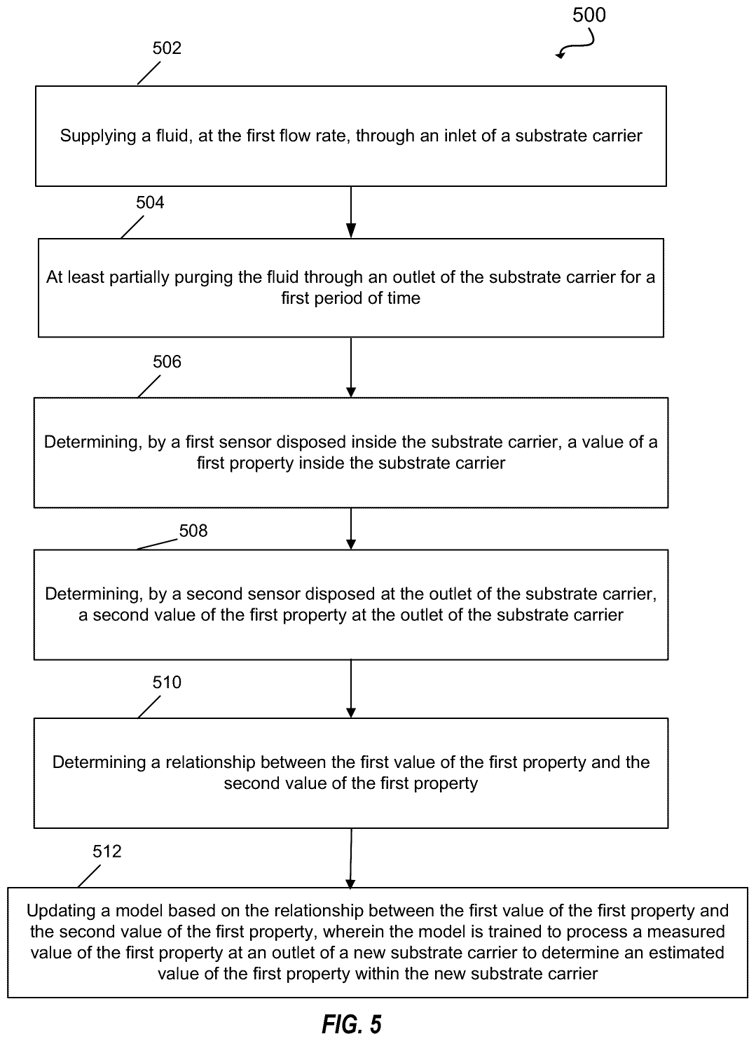

System controller 140 can be and/or include a computing device such as a personal computer, a server computer, a programmable logic controller (PLC), a microcontroller, and so on. System controller 140 can include one or more processing devices, which can be general-purpose processing devices such as a microprocessor, central processing unit, or the like. More particularly, the processing device can be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device can also be one or more special-purpose processing devices such as an application specific integrated circuit (ASIC), a field programmable gate array (FPGA), a digital signal processor (DSP), network processor, or the like. System controller 140 can include a data storage device (e.g., one or more disk drives and/or solid state drives), a main memory, a static memory, a network interface, and/or other components. System controller 140 can execute instructions to perform any one or more of the methodologies and/or embodiments described herein. The instructions can be stored on a computer readable storage medium, which can include the main memory, static memory, secondary storage and/or processing device (during execution of the instructions). System controller 140 can include an environmental controller configured to control an environment (e.g., pressure, moisture level, vacuum level, etc.) within factory interface 106 and/or within substrate carrier 200. In embodiments, execution of the instructions by system controller 140 causes system controller to perform the methods of one or more of FIGS. 4 and 5. System controller 140 can also be configured to permit entry and display of data, operating commands, and the like by a human operator.

System controller 140 can include suitable processor, memory, and electronic components for receiving inputs from various sensors and for controlling one or more valves, doors, etc. to control the environmental conditions within the EFEM 104 and/or substrate carrier 200. System controller 140 can include an environmental control system that can, in one or more embodiments, monitor relative humidity (RH) within substrate carrier 200 by sensing RH in exhaust line 146 from sensor 116 and estimating a RH within substrate carrier 200 based on the measured RH in exhaust line 146. Any suitable type of sensor that measures relative humidity can be used, such as a capacitive-type sensor. The RH in substrate carrier 200 can be lowered by flowing a suitable amount of purge gas from a purge gas supply of the environmental control system into substrate carrier 200. In some embodiments, compressed bulk inert gases having low H₂O levels (e.g., purity≥99.9995%, H₂O≤5 ppm) can be used as the purge gas supply in the environmental control system, for example. Other suitably low H₂O levels can be used.

In another aspect, the sensor(s) 116 can measure a plurality of environmental conditions within substrate carrier 200 based on measurements of sensors 116 in exhaust line 146. For example, in some embodiments, the sensor(s) can measure a relative humidity value. In one or more embodiments, a pre-defined reference relative humidity value can be less than 1000 ppm moisture, less than 500 ppm moisture, or even less than 100 ppm moisture, depending upon the level of moisture that is tolerable for the particular process being carried out in the system 100 or particular substrates exposed to the environment of one or both of the factory interface chambers.

The environmental monitor can also estimate a level of oxygen (O₂) within substrate carrier 200. In some embodiments, a control signal from the system controller 140 to the environmental control apparatus initiating a flow of a suitable amount of purge gas from the purge gas supply into the substrate carrier 200 can take place to control the level of oxygen (O₂) to below a threshold O₂ value. In one or more embodiments, the threshold O₂ value can be less than 50 ppm, less than 10 ppm, or even less than 5 ppm, depending upon the level of O₂ that is tolerable (not affecting quality) for the particular process being carried out in the system 100 or particular substrates exposed to the environment of one of the factory interface chambers. In some embodiments, the sensor(s) can sense the level of oxygen in substrate carrier 200 to ensure it is below a safe threshold level to allow entry of substrates from substrate carrier 200 into the EFEM 104.

The sensor(s) 116 can further measure the absolute or relative pressure within the substrate carrier 200. In some embodiments, the system controller 140 can control the amount of flow of purge gas from a purge gas supply into substrate carrier 200 to control the pressure in the substrate carrier 200. The sensors 116 may also measure temperature, particles, volatile compounds, and/or other properties of an exhaust, which may be used to estimate values of those properties within substrate carrier 200, as is described in greater detail below.

In the embodiments shown herein, the system controller 140 can include a processor, memory, and peripheral components configured to receive control inputs (e.g., relative humidity and/or oxygen) from the sensor(s) and to execute a closed loop or other suitable control scheme. In one embodiment, the control scheme can change a flow rate of the purge gas being introduced into the substrate carrier 200, an amount of time to flow purge gas into substrate carrier 200 before opening a substrate carrier door 132, etc. to achieve a predetermined environmental condition therein.

In embodiments, controller 140 may receive a measurement of a property of an exhaust from substrate carrier 200, and process the measurement to estimate a value of the property within substrate carrier 200. In some embodiments, controller 140 includes a trained machine learning model that is used to process data from sensor(s) 116 and to output estimates of RH, oxygen level, particle level, etc. within substrate carrier 200. When controller 140 determines that environmental conditions within substrate carrier 200 satisfy one or more criteria, controller 140 may cause door opener 114 to open substrate carrier door 132. Substrates may then be retrieved from substrate carrier 200 by a robot of EFEM 104 for processing.

FIG. 1B illustrates a schematic view of an example system 100 (e.g., which may correspond to system 100 of FIG. 1A) according to one or more embodiments of the disclosure. The system 100 includes a FOUP 200 that may be mounted on a docking station 120 that may lock the FOUP 200 in place. The docking station 120 may include a dock or other support for FOUP 200. FOUP 200 may be used to safely store and transport substrates (e.g., silicon wafers) while maintaining a controlled and clean environment. FOUP 200 may also be used to prevent contamination and damage to substrates during various stages of the semiconductor manufacturing process or display manufacturing process.

System 100 may include additional automated handling systems, such as robots or conveyors (e.g., of an EFEM), that can load and unload substrates without exposing them to the external environment. FOUP 200 may be equipped with identification systems, such as radio-frequency identification (RFID) tags, barcodes, or other tracking mechanisms. These systems help in tracking the contents of the FOUP, including information about the wafers, their manufacturing history, and their location within the cleanroom or manufacturing facility. FOUP 200 may be compatible with various semiconductor manufacturing equipment, such as lithography machines, etching, deposition tools, and more. FOUP 200 may be transported using automated guided vehicles (AGVs) or other material handling systems within the cleanroom or semiconductor manufacturing facility.

In some embodiments, system 100 may be used to analyze and monitor an inside environment of FOUP 200. Initially, the analysis may include determining the amount of relative humidity (RH) inside the FOUP 200. In some embodiments, the analysis may include determining the amount of a gas (e.g., oxygen) inside the FOUP 200. In some embodiments, the analysis may include determining the amount of total volatile organic compounds (TVOC) inside the FOUP 200. In some embodiments, the analysis may include determining the number of particles (e.g., aerosol) inside the FOUP 200. In some embodiments, the analysis may include determining the temperature inside the FOUP 200. In some embodiments, the analysis may include determining the amount of two or more of the above parameters inside the FOUP 200.

FOUP 200 may include one or more inlets 118 for a fluid (e.g., nitrogen or clean dry air (CDA)) to flow therethrough and into the FOUP 200, and one or more outlets 126 to purge the fluid out of the FOUP 200. A purge kit 132 (e.g., a fluid cylinder) including a pressurized fluid (e.g., nitrogen or clean dry air (CDA)) may be coupled to the inlet 118 to supply the fluid to the FOUP 200. One or more sensors 116 (e.g., MEMS sensors) may be coupled to the outlet 126 to measure a parameter outside the FOUP 200. For example, the one or more sensors 126 may be installed in an outlet pipe of the FOUP where a gas may be purged. The one or more sensors 126 may be connected in series or in parallel to measure the one or more parameters. For example, an RH sensor may be coupled with at least one of a gas sensor, a temperature sensor, a chemical senor, etc. One or more sensors 138 (e.g., MEMS sensors) may be used to measure one or more parameters inside the FOUP 200. The one or more sensors 138 may be installed on one or more slots (not shown) of the FOUP 200. For example, one sensor may be disposed on the first slot of the FOUP, one sensor may be disposed on the 13^{th} slot of the FOUP, and one sensor may be disposed on the 25^{th} slot of the FOUP. Additional sensors may be deployed in other areas inside the FOUP 200. Alternatively, only one sensor may be installed inside the FOUP (e.g., a single sensor may be installed on the 13^{th} slot of the FOUP). The one or more sensors 138, 116 may include a RH sensor, a gas sensor, a temperature sensor, a chemical sensor, or combinations thereof. In some embodiments, the FOUP 200 may have one or more RH sensors and one or more gas sensors. Sensors 138, 116 may be coupled to a microcontroller 140, which may receive measurement values from sensors 138, 116. Valves 134, 136 may also be coupled to the microcontroller 140, which may control a flow rate of the pressurized fluid into the FOUP 200 or into the sensor 116.

System 100 may further include an input line 110 to supply the fluid (e.g., nitrogen or CDA), and an exhaust line 146, which may be connected to one or more motors and may be used to purge the fluid out of the FOUP 200. Valve 134 may be used to control a flow rate of the pressurized fluid going into the FOUP 200. Valve 136 may be used to control a flow rate of the pressurized fluid going directly to the sensor 116. Valve 136 may be used to calibrate a reading on sensor 116. For example, after completing analysis of a parameter, valve 134 may be closed and valve 136 may be opened so sensor 116 is down to equilibrium state (e.g., 0% RH, 0% oxygen, desired room temperature, etc.).

When a FOUP 200 is received (e.g., from another location within the cleanroom or another manufacturing facility), one or more parameters relating to the inside environment of the FOUP 200 is measured using the one or more sensors inside the FOUP 200. For example, an initial RH value may be measured, an initial oxygen level may be measured, an initial TVOC content may be measured, an initial temperature, and/or an initial particle (e.g., aerosol particle) count may be measured, and time elapsed since the last time the front door of the FOUP was closed (e.g., age) may be recorded. Since the RH and/or other parameters inside the FOUP change over time, age is recorded for comparing FOUPs with same/different ages. Age may refer to an amount of time that a lot is contained within a FOUP. A database (e.g., a metadata table) may be generated including the different values for RH, oxygen, TVOC, temperature, and particle count over different ages.

In some embodiments, a test gas 125 may be supplied to the FOUP 200 at a first flow rate. The test gas 125 (e.g., nitrogen (N₂) or clean dry air (CDA)) may be supplied through one or more inlets 118 provided in the FOUP 200. After supplying the test gas 125 for a period of time at the first flow rate, the test gas 125 may be at least partially purged through one or more outlets 126 of the FOUP 200. Alternatively, the test gas 125 may be purged as the test gas 125 is being supplied to the FOUP 200 at the first flow rate. As the test gas 125 is being purged from the FOUP 200, the one or more sensors 116 on the outside of the FOUP 200 measure one or more parameters (e.g., RH, oxygen, TVOC, temperature, and/or particle count) in real-time. The outside measurements for each of the parameters are plotted against the inside measurements for the same parameters over a period to determine a relationship between the outside measurement and the inside measured of the parameter. For example, the relationship between the outside measurement and the inside measurement may be expressed as an equation having a gradient, a y-intercept, and/or other factors. The relationship between the outside measurement and the inside measurement may vary based on the initial reading of the parameter (e.g., RH, oxygen, TVOC, temperature, and/or particle count) and the age (e.g., time elapsed since the last time the front door of the FOUP was closed) of the FOUP. The initial values for the parameters, the real-times values, and the age of the FOUP may be recorded in the database, and historical values (e.g., recorded over a period of time for different FOUPs) may be used to train a machine learning model, which may then output an inside measurement based on the outside measurement and age of the FOUP.

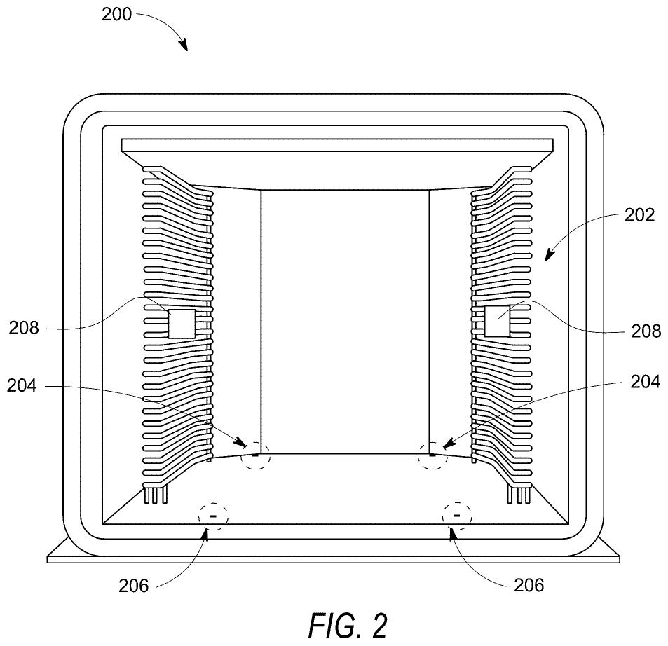

FIG. 2 illustrates a schematic view of a front opening unified pod (FOUP) 200 according to one or more embodiments of the disclosure. FOUP 200 may have a front-opening design including a front door (not shown) and a main body. The front door may be used to access the wafers inside, and the FOUP may be designed to maintain a seal to prevent particles and contaminants from entering the FOUP when the front door is closed. FOUP 200 may include one or more air filtration systems to remove particles and protect the wafers from contamination.

FOUP 200 may include one or more inlets 204 for a fluid (e.g., nitrogen or clean dry air (CDA)) to flow therethrough and into the FOUP 200, and one or more outlets 206 to purge the fluid out of the FOUP 200. A purge kit (e.g., a fluid cylinder) including a pressurized fluid (e.g., nitrogen or clean dry air (CDA)) may be coupled to the inlet 204 to supply the fluid to the FOUP 200. One or more sensors 208 (e.g., MEMS sensors) may be used to measure one or more parameters inside the FOUP 200. The one or more sensors 208 may be installed on one or more slots 202 of the FOUP 200. For example, one sensor may be disposed on the first slot of the FOUP, one sensor may be disposed on the 13^{th} slot of the FOUP, and one sensor may be disposed on the 25^{th} slot of the FOUP. Additional sensors may be deployed in other areas inside the FOUP 200. Alternatively, only one sensor may be installed inside the FOUP (e.g., a single sensor may be installed on the 13^{th} slot of the FOUP). The one or more sensors 208 may include a RH sensor, a gas sensor, a temperature sensor, a chemical sensor, or combinations thereof. In some embodiments, the FOUP 200 may have one or more RH sensors and one or more gas sensors. Sensors 208 may be coupled to a microcontroller (not shown), which may receive measurement values from sensors 208.

When a front opening unified pod (FOUP) 200 is received at a process interface (e.g., etching, deposition, lithography, or other tools), a fluid (e.g., a test gas such as nitrogen) may be supplied through one or more inlets 204 at a first flow rate. After supplying the test gas for a period of time at the first flow rate, the test gas may be at least partially purged through one or more outlets 206 of the FOUP. Alternatively, the test gas may be purged as the test gas is being supplied to the FOUP at the first flow rate. As the test gas is being purged from the FOUP 200, the one or more sensors on the outside of the FOUP measure the one or more parameters (e.g., RH, oxygen, TVOC, temperature, and/or particle count) in real-time. The outside measurements may be input into the machine learning model, and the machine learning model may output an inside measurement based on the outside measurement and age (e.g., time elapsed since the last time the front door was closed) of the FOUP. If the derived value of the inside measurement is greater than a threshold value, then an alert may be generated at a load port unit (LPU) of a processing chamber including the FOUP, and the FOUP may be further treated (e.g., with an inert gas) to remove any residual RH inside the FOUP. However, if the derived value of the inside measurement is within an acceptable range (e.g., equal to or lesser than a threshold value), then the FOUP is loaded onto the LPU for further processing.

In some embodiments, a microcontroller (e.g., microcontroller 140) may determine a first RH in the FOUP at a first time, determine a second RH in the FOUP at a second time, and determine a leak rate of the FOUP based on the difference between the first RH and the second RH and time elapsed between the first time and the second time. In some embodiments, the microcontroller may determine that the RH inside the FOUP has dropped below a RH threshold, and open a door of the FOUP in response so that one or more substrates (e.g., semiconductor wafers) may be loaded onto or unloaded from the FOUP.

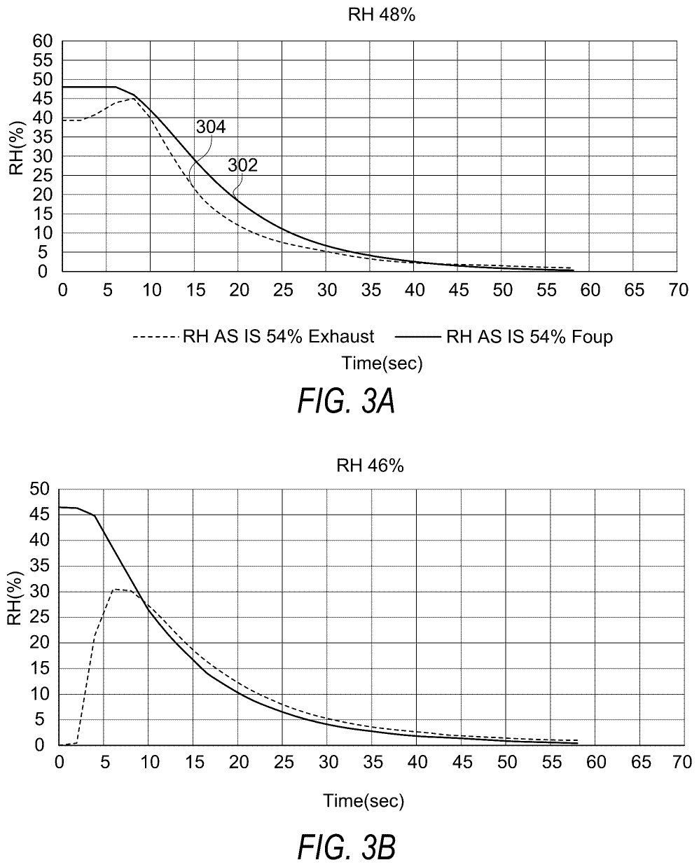

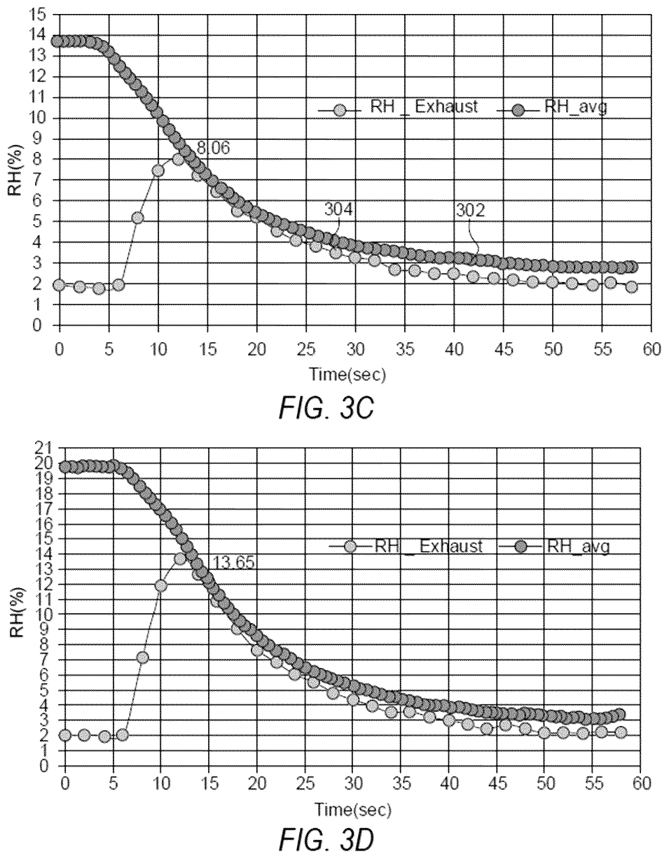

FIGS. 3A-3D illustrate different charts comparing relative humidity (RH) measured inside the FOUP 302 versus RH measured at the outlet or exhaust port 304 over a period of time. For example, FIG. 3A illustrates a curve with an inside RH 302 of 48%. As the test gas is being pumped into the FOUP, the outside measurement aligns with the inside measurement, and both values gradually go down to 0% RH after a period of time (e.g., because of the nitrogen or CDA being pumped continuously). The time required for the curves to reach 0% RH may be dependent on the age of the FOUP. Similarly, FIG. 3B illustrates a curve with an inside RH 302 of 46%. As the test gas is being pumped into the FOUP, the outside measurement aligns with the inside measurement, and both values gradually go down to 0% RH after a period of time. Similarly, FIG. 3C illustrates a curve with an inside RH 302 of 14%. As the test gas is being pumped into the FOUP, the outside measurement aligns with the inside measurement, and both values gradually go down to 0% RH after a period of time. Although the time required to reach 0% RH is the same as FIG. 3B, the flow rate in FIG. 3C is lower than that in FIG. 3A and FIG. 3B. Similarly, FIG. 3D illustrates a curve with an inside RH 302 of 20%. As the test gas is being pumped into the FOUP, the outside measurement aligns with the inside measurement, and both values gradually go down to 0% RH after a period of time. Although the time required to reach 0% RH is the same as FIG. 3B, the flow rate in FIG. 3D is lower than that in FIG. 3A and FIG. 3B.

In some embodiments, the microcontroller may determine a first temperature inside the FOUP at a first time, determine a second temperature inside the FOUP at a second time, and determine a rate of temperature change based on the difference between the first temperature and the second temperature and time elapsed between the first time and the second time. In some embodiments, the microcontroller may determine that the temperature inside the FOUP has dropped below a temperature threshold, and open a door of the FOUP in response so that one or more substrates (e.g., semiconductor wafers) may be loaded onto or unloaded from the FOUP.

FIG. 4 illustrates a flowchart of a method 400 for monitoring inside parameters of a substrate carrier according to one or more embodiments of the disclosure. At operation 402, the method 400 may include connecting a front opening unified pod (FOUP) at a process interface (e.g., etching, deposition, lithography, or other tools) of a load port unit (LPU). The interface may have one or more position sensors to ensure proper placement and locking of the FOUP to the process interface. At operation 404, the method may include opening a first valve (e.g., valve 134) of the PLU and optionally closing a second valve (e.g., valve 136) so as to allow a fluid (e.g., nitrogen gas) to flow into the FOUP. At operation 406, the method may include supplying a pressurized fluid at a first flow rate through one or more inlets of the FOUP for a first period of time. At operation 408, after supplying the test gas for a period of time at the first flow rate, the first valve is closed and the test gas may be at least partially purged through one or more outlets of the FOUP. Alternatively, the test gas may be purged as the test gas is being supplied to the FOUP at the first flow rate. As the test gas is being purged from the FOUP, at operation 410, one or more sensors on the outside of the FOUP measure the one or more parameters (e.g., RH, oxygen, TVOC, temperature, and/or particle count) in real-time. At operation 412, the outside measurements may be input into the machine learning model, and the machine learning model may output an inside measurement based on the outside measurement and age (e.g., time elapsed since the last time the front door was closed) of the FOUP, and a predetermined relationship between the outside measurement and the inside measurement. At operation 414, if the derived value of the inside measurement is greater than a threshold value, then an alert may be generated at a load port unit (LPU) of a processing chamber including the FOUP, and the FOUP may be further treated (e.g., with an inert gas) to remove any residual RH inside the FOUP. However, if the derived value of the inside measurement is within an acceptable range (e.g., equal to or lesser than a threshold value), then at operation 416, the FOUP door may be opened into an EFEM for further processing of substrates within the FOUP.

In some embodiments, method 400 includes determining a first RH in the FOUP at a first time, determining a second RH in the FOUP at a second time, and determining a leak rate of the FOUP based on the difference between the first RH and the second RH and time elapsed between the first time and the second time. In some embodiments, if a leak rate of a FOUP is determined to be above a threshold, then the FOUP may be flagged for maintenance and/or may be taken out of service. In some embodiments, the method 400 includes determining that the RH inside the FOUP has dropped below a RH threshold, and opening a door of the FOUP in response so that one or more substrates (e.g., semiconductor wafers) may be loaded onto or unloaded from the FOUP.

FIG. 5 illustrates a flowchart of a method 500 for monitoring inside parameters of a substrate carrier according to one or more embodiments of the disclosure. The method 500 may include receiving a front opening unified pod (FOUP) at a process interface (e.g., etching, deposition, lithography, or other tools) of a load port unit (LPU). The interface may have one or more position sensors to ensure proper placement and locking of the FOUP to the process interface. At operation 502, the method may include supplying a pressurized fluid (e.g., nitrogen gas) at a first flow rate through one or more inlets of the FOUP for a first period of time. At operation 504, after supplying the test gas for a period of time at the first flow rate, the test gas may be at least partially purged through one or more outlets of the FOUP. Alternatively, the test gas may be purged as the test gas is being supplied to the FOUP at the first flow rate. As the test gas is being purged from the FOUP, at operation 506, the method may include determining, by a first sensor disposed inside the substrate carrier, a value of a first property inside the substrate carrier (e.g., oxygen, TVOC, temperature, and/or particle count) in real-time. At operation 508, the method may include determining, by a second sensor disposed at the outlet of the substrate carrier, a second value of the first property at the outlet of the substrate carrier. At operation 510, the method may include determining a relationship between the first value of the first property and the second value of the first property. At operation 512, the method may include updating a model based on the relationship between the first value of the first property and the second value of the first property. The model may be trained to process a measured value of the first property at an outlet of a new substrate carrier to determine an estimated value of the first property within the new substrate carrier. When the new substrate carrier is received, the outside measurements may be input into the machine learning model, and the machine learning model may output an inside measurement based on the outside measurement and age (e.g., time elapsed since the last time the front door was closed) of the FOUP, and a predetermined relationship between the outside measurement and the inside measurement. If the derived value of the inside measurement is greater than a threshold value, then an alert may be generated at a load port unit (LPU) of a processing chamber including the FOUP, and the FOUP may be further treated (e.g., with an inert gas) to remove any residual RH inside the FOUP. However, if the derived value of the inside measurement is within an acceptable range (e.g., equal to or lesser than a threshold value), the FOUP is loaded onto the LPU for further processing.

In some embodiments, the method 500 may include determining a first temperature inside the FOUP at a first time, determining a second temperature inside the FOUP at a second time, and determining a rate of temperature change based on the difference between the first temperature and the second temperature and time elapsed between the first time and the second time. In some embodiments, the method 500 may include determining that the temperature inside the FOUP has dropped below a temperature threshold, and opening a door of the FOUP in response so that one or more substrates (e.g., semiconductor wafers) may be loaded onto or unloaded from the FOUP.

The devices, systems, and methods disclosed herein provide machine learning platforms for monitoring an inside environment of a substrate carrier (e.g., a FOUP). The processing logic trains a machine learning model with data input including the features to generate a trained machine learning model. The trained machine learning model is capable of generating one or more outputs indicative of one or more corrective actions to be performed in the EFEM.

In some embodiments, a machine learning platform receives the historical data and outputs the trained machine learning model. In some embodiments, the machine learning platform receives user input specifying types of feature definitions (e.g., age of the FOUP, measured values, flow rate of the fluids, etc.). In some embodiments, the machine learning platform receives user input specifying type of target output (e.g., model definition). In some embodiments, the machine learning platform provides a user interface (e.g., graphical user interface) to receive the historical data, feature definitions, and/or model definitions.

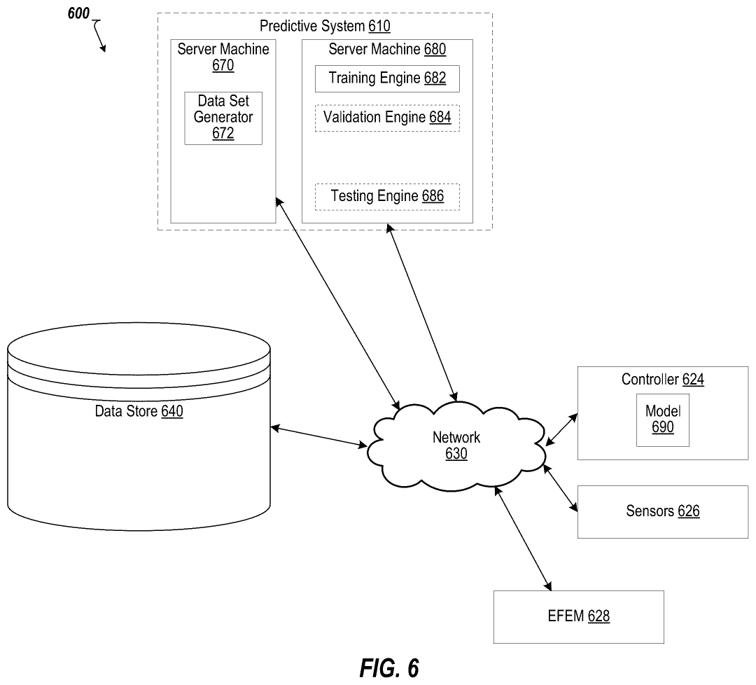

FIG. 6 is a block diagram illustrating an exemplary system 600 (exemplary system architecture), according to certain embodiments. The system 600 includes an EFEM 628, a controller 624, sensors 626, a predictive server, and a data store 640. In some embodiments, the predictive server is part of a predictive system 610. In some embodiments, the predictive system 610 further includes a server machine 680. One or more of the components (e.g., controller 624, sensors 626, etc.) of system 600 may be part of the same substrate processing facility.

In some embodiments, one or more of the EFEM 628, controller 624, sensors 626, predictive system 610, data store 640, and/or server machine 680 are coupled to each other via a network 630 for generating predictive data to perform corrective actions. In some embodiments, network 630 is a public network that provides EFEM 628 with access to the predictive server, data store 640, and other publicly available computing devices. In some embodiments, network 630 is a private network that provides EFEM 628 access to manufacturing equipment, sensors 626, data store 640, and other privately available computing devices. In some embodiments, network 630 includes one or more Wide Area Networks (WANs), Local Area Networks (LANs), wired networks (e.g., Ethernet network), wireless networks (e.g., an 802.11 network or a Wi-Fi network), cellular networks (e.g., a Long Term Evolution (LTE) network), routers, hubs, switches, server computers, cloud computing networks, and/or a combination thereof.

In some embodiments, the client device includes a computing device such as Personal Computers (PCs), laptops, mobile phones, smart phones, tablet computers, netbook computers, etc. In some embodiments, the client device includes one or more parameters component. Client device includes an operating system that allows users to one or more of generate, view, or edit data (e.g., indication associated with manufacturing equipment, corrective actions associated with substrate processing facility, etc.).

In some embodiments, controller 624 may have the trained ML model stored on it. The controller 624 may receive sensor data from sensors, process the sensor data using the model, and control the EFEM based on the output of model. For training the ML model, the server machine 670 may include data about the properties inside and outside of the FOUP, and server machine 680 may use that data to train the ML model, validate it, etc.

In some embodiments, the predictive system 610 and server machine 680 each include one or more computing devices such as a rackmount server, a router computer, a server computer, a personal computer, a mainframe computer, a laptop computer, a tablet computer, a desktop computer, Graphics Processing Unit (GPU), accelerator Application-Specific Integrated Circuit (ASIC) (e.g., Tensor Processing Unit (TPU)), etc.

Server machine 680 includes a training engine 682, a validation engine 684, selection engine 685, and/or a testing engine 686. In some embodiments, an engine (e.g., training engine 682, a validation engine 684, selection engine 685, and a testing engine 686) refers to hardware (e.g., circuitry, dedicated logic, programmable logic, microcode, processing device, etc.), software (such as instructions run on a processing device, a general purpose computer system, or a dedicated machine), firmware, microcode, or a combination thereof. The training engine 682 is capable of training a machine learning model 690 using one or more sets of features associated with the training set from data set generator 6762. In some embodiments, the training engine 682 generates multiple trained machine learning models 690, where each trained machine learning model 690 corresponds to a distinct set of features of the training set (e.g., based on facility data from a distinct set of sensors).

The trained machine learning model(s) 690 may process sensor data from one or more sensors and output the % RH, % oxygen, temperature, TVOC count, particle count, etc. inside the substrate carrier. In one embodiment, trained ML model 690. In some instances, multiple machine learning models are used, where each machine learning model to receive sensor data from one or more sensors and output the % RH, % oxygen, temperature, TVOC count, particle count, etc. inside the substrate carrier.

The one or more trained machine learning models 690 may be support vector machines, random forest models, Bayesian classifiers, regression models, neural networks such as deep neural networks or convolutional neural networks, generative models, and so on. ML models 690 may be trained for measured values of one or more parameters such as RH, temperature, oxygen, TVOC, particle count, and so on. Some ML models 690 may be or include generative models, such as generative adversarial networks (GANs). ML models 690 may include machine learning models trained using supervised training and/or ML models trained using unsupervised learning. In some embodiments, one or more ML models 690 are reinforcement learning models.

Artificial neural networks (e.g., deep neural networks and convolutional neural networks) generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g., classification outputs). Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes; and the fourth layer may recognize that the image contains a face. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

Training of a neural network may be achieved in a supervised learning manner, which involves feeding a training dataset including labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available. To train the one or more machine learning models 690, a training dataset (or multiple training datasets, one for each of the machine learning models to be trained) containing hundreds, thousands, tens of thousands, hundreds of thousands or more images should be used to form a training dataset.

In some embodiments, a “user” is represented as a single individual. However, other embodiments of the disclosure encompass a “user” being an entity controlled by a plurality of users and/or an automated source. In some examples, a set of individual users federated as a group of administrators is considered a “user.”

Although embodiments of the disclosure are discussed in terms of generating predictive data to perform one or more parameters in manufacturing facilities (e.g., substrate processing facilities), in some embodiments, the disclosure can also be generally applied to causing corrective actions for setting the % RH, the temperature, or % oxygen inside the FOUP.

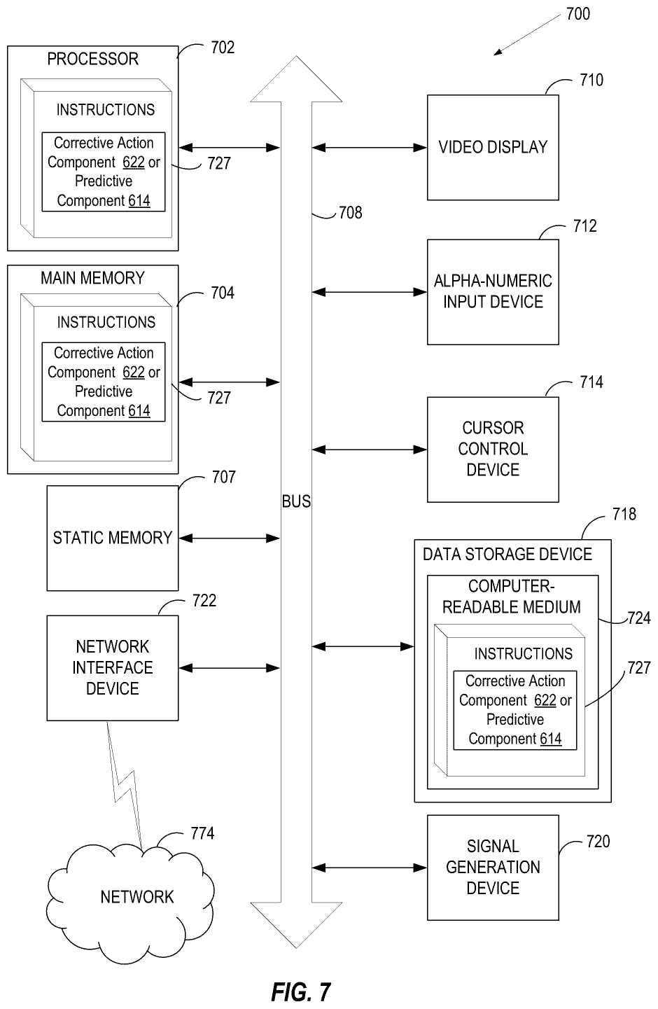

FIG. 7 is a block diagram illustrating a computer system 700, according to certain embodiments. In some embodiments, the computer system 700 is one or more of EFEM 628, predictive system 610, server machine 680, or predictive system 610. In some embodiments, computer system 700 may form a part of a FOUP (e.g., FOUP 200), a part of an EFEM, and/or a part of a LPU.

In some embodiments, computer system 700 is connected (e.g., via a network, such as a Local Area Network (LAN), an intranet, an extranet, or the Internet) to other computer systems. In some embodiments, computer system 700 operates in the capacity of a server or a client computer in a client-server environment, or as a peer computer in a peer-to-peer or distributed network environment. In some embodiments, computer system 700 is provided by a personal computer (PC), a tablet PC, a Set-Top Box (STB), a Personal Digital Assistant (PDA), a cellular telephone, a web appliance, a server, a network router, switch or bridge, or any device capable of executing a set of instructions (sequential or otherwise) that specify actions to be taken by that device. Further, the term “computer” shall include any collection of computers that individually or jointly execute a set (or multiple sets) of instructions to perform any one or more of the methods described herein.

In a further aspect, the computer system 700 includes a processing device 702, a volatile memory 704 (e.g., Random Access Memory (RAM)), a non-volatile memory 707 (e.g., Read-Only Memory (ROM) or Electrically-Erasable Programmable ROM (EEPROM)), and a data storage device 717, which communicate with each other via a bus 708.

In some embodiments, processing device 702 is provided by one or more processors such as a general purpose processor (such as, for example, a Complex Instruction Set Computing (CISC) microprocessor, a Reduced Instruction Set Computing (RISC) microprocessor, a Very Long Instruction Word (VLIW) microprocessor, a microprocessor implementing other types of instruction sets, or a microprocessor implementing a combination of types of instruction sets) or a specialized processor (such as, for example, an Application Specific Integrated Circuit (ASIC), a Field Programmable Gate Array (FPGA), a Digital Signal Processor (DSP), or a network processor).

In some embodiments, computer system 700 further includes a network interface device 722 (e.g., coupled to network 774). In some embodiments, computer system 700 also includes a video display unit 710 (e.g., an LCD), an alphanumeric input device 712 (e.g., a keyboard), a cursor control device 714 (e.g., a mouse), and a signal generation device 720.

In some implementations, data storage device 717 includes a non-transitory computer-readable storage medium 724 on which store instructions 727 encoding any one or more of the methods or functions described herein, including instructions encoding components of FIG. 1 (e.g., corrective action component 722, predictive component 714, etc.) and for implementing methods described herein (e.g., one or more of methods 400, 500).

In some embodiments, instructions 727 also reside, completely or partially, within volatile memory 704 and/or within processing device 702 during execution thereof by computer system 700, hence, in some embodiments, volatile memory 704 and processing device 702 also constitute machine-readable storage media.

While computer-readable storage medium 724 is shown in the illustrative examples as a single medium, the term “computer-readable storage medium” shall include a single medium or multiple media (e.g., a centralized or distributed database, and/or associated caches and servers) that store the one or more sets of executable instructions. The term “computer-readable storage medium” shall also include any tangible medium that is capable of storing or encoding a set of instructions for execution by a computer that cause the computer to perform any one or more of the methods described herein. The term “computer-readable storage medium” shall include, but not be limited to, solid-state memories, optical media, and magnetic media.

In some embodiments, the methods, components, and features described herein are implemented by discrete hardware components or are integrated in the functionality of other hardware components such as ASICS, FPGAs, DSPs or similar devices. In some embodiments, the methods, components, and features are implemented by firmware modules or functional circuitry within hardware devices. In some embodiments, the methods, components, and features are implemented in any combination of hardware devices and computer program components, or in computer programs.

Unless specifically stated otherwise, terms such as “identifying,” “generating,” “training,” “storing,” “receiving,” “determining,” “causing,” “providing,” “obtaining,” “updating,” “re-training,” or the like, refer to actions and processes performed or implemented by computer systems that manipulates and transforms data represented as physical (electronic) quantities within the computer system registers and memories into other data similarly represented as physical quantities within the computer system memories or registers or other such information storage, transmission or display devices. In some embodiments, the terms “first,” “second,” “third,” “fourth,” etc. as used herein are meant as labels to distinguish among different elements and do not have an ordinal meaning according to their numerical designation.

Examples described herein also relate to an apparatus for performing the methods described herein. In some embodiments, this apparatus is specially constructed for performing the methods described herein, or includes a general purpose computer system selectively programmed by a computer program stored in the computer system. Such a computer program is stored in a computer-readable tangible storage medium.

The methods and illustrative examples described herein are not inherently related to any particular computer or other apparatus. In some embodiments, various general purpose systems are used in accordance with the teachings described herein. In some embodiments, a more specialized apparatus is constructed to perform methods described herein and/or each of their individual functions, routines, subroutines, or operations. Examples of the structure for a variety of these systems are set forth in the description above.

The above description is intended to be illustrative, and not restrictive. Although the present disclosure has been described with references to specific illustrative examples and implementations, it will be recognized that the present disclosure is not limited to the examples and implementations described. The scope of the disclosure should be determined with reference to the following claims, along with the full scope of equivalents to which the claims are entitled.

Reference throughout this specification to, for example, “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

As used herein, the singular forms “a,” “an,” and “the” include plural references unless the context clearly indicates otherwise. Thus, for example, reference to “a robot arm” includes a single robot arm as well as more than one robot arm.

As used herein, the term “about” in connection with a measured quantity, refers to the normal variations in that measured quantity as expected by one of ordinary skill in the art in making the measurement and exercising a level of care commensurate with the objective of measurement and the precision of the measuring equipment. In certain embodiments, the term “about” includes the recited number ±10%, such that “about 10” would include from 9 to 11.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term “at least about” includes the recited number minus 10% and any quantity that is higher such that “at least about 10” would include 9 and anything greater than 9. This term can also be expressed as “about 10 or more.” Similarly, the term “less than about” typically includes the recited number plus 10% and any quantity that is lower such that “less than about 10” would include 11 and anything less than 11. This term can also be expressed as “about 10 or less.”

The foregoing description discloses example embodiments of the disclosure. Modifications of the above-disclosed assemblies, apparatus, and methods which fall within the scope of the disclosure will be readily apparent to those of ordinary skill in the art. Accordingly, while the present disclosure has been disclosed in connection with example embodiments, it should be understood that other embodiments may fall within the scope of the disclosure, as defined by the following claims.

What is claimed is:

1. A method comprising:
receiving a first substrate carrier;
supplying a fluid, at a first flow rate, through an inlet of the first substrate carrier;
at least partially purging the fluid through an outlet of the first substrate carrier for a first period of time;
measuring, using a first sensor disposed at the outlet, a first value of a first property of an exhaust from the substrate carrier at the outlet of the first substrate carrier; and
determining, based at least in part on the first value of the first property, a second value of the first property inside the first substrate carrier.

2. The method of claim 1, wherein determining the second value of the first property inside the first substrate carrier comprises inputting the first value of the first property into a model that outputs the second value of the first property.

3. The method of claim 2, further comprising training the model, the training comprising:
supplying the fluid, at the first flow rate, through an inlet of a second substrate carrier;
at least partially purging the fluid through an outlet of the second substrate carrier for the first period of time;
determining, by a second sensor disposed inside the second substrate carrier, a third value of the first property inside the second substrate carrier;
determining, by a third sensor disposed at the outlet of the second substrate carrier, a fourth value of the first property at the outlet of the second substrate carrier;
determining a relationship between the third value of the first property and the fourth value of the first property; and
updating the model based on the relationship between the third value of the first property and the fourth value of the first property.

4. The method of claim 2, wherein the model comprises a trained machine learning model.

5. The method of claim 1, further comprising:
determining the second value of the first property inside the first substrate carrier is greater than a threshold value; and
transmitting a message to a load port unit (LPU) of a processing chamber comprising the first substrate carrier.

6. The method of claim 1, further comprising:
determining a third value of the first property in the first substrate carrier measured at a first time;
determining a first difference between the second value of the first property and the third value of the first property;
determining an elapsed time between the first time and a second time at which the second value of the first property was measured; and
determining a leak rate of the first substrate carrier based on the first difference and the elapsed time.

7. The method of claim 1, wherein the first property comprises relative humidity (RH), the method further comprising:
determining when the second value of the first property drops below a RH threshold; and
opening a door of the first substrate carrier responsive to determining that the second value of the first property has dropped below the RH threshold.

8. The method of claim 1, wherein the first property comprises an amount of a first gas, wherein the first gas comprises at least one of oxygen, aerosol particles, or total volatile organic compounds (TVOC).

9. The method of claim 8, further comprising:
determining the second amount of the first gas inside the first substrate carrier is greater than a threshold value; and
transmitting a message to a load port unit (LPU) of a processing chamber comprising the first substrate carrier.

10. The method of claim 1, wherein the first property comprises temperature, the method further comprising:
determining when the second value of the first property is within a threshold range; and
opening a door of the first substrate carrier responsive to determining that the second value of the first property is within the threshold range.

11. The method of claim 10, further comprising:
determining the second value inside the first substrate carrier is greater than a threshold value; and
transmitting an alert to a load port unit (LPU) of a processing chamber comprising the first substrate carrier.

12. The method of claim 1, wherein the first substrate carrier comprises a first FOUP, wherein the first sensor comprises a MEMS sensor, wherein the first property comprises relative humidity (RH), wherein the first value comprises a RH measured at the outlet of the first substrate carrier, or wherein the second value comprises a RH inside the first substrate carrier.

13. A system comprising:
a first front opening unified pod (FOUP), the first FOUP having an inlet to receive a fluid at a first flow rate and an outlet to at least partially purge the fluid for a first period of time;
a first sensor disposed at the outlet, the first sensor to determine a first relative humidity (RH) or a first amount of a first gas at the outlet of the first FOUP; and
a controller operatively coupled to the first sensor, the controller configured to determine, based at least in part on the first RH or the first amount of the first gas at the outlet, a second RH or a second amount of the first gas inside the first FOUP.

14. The system of claim 13, wherein determining the second RH inside the first FOUP comprises inputting the first RH into a model that outputs the second RH.

15. The system of claim 14, wherein the model is trained by:
supplying the fluid, at the first flow rate, through an inlet of a second FOUP;
at least partially purging the fluid through an outlet of the second FOUP for a second period of time;
determining, by a second sensor disposed inside the second FOUP, a third RH inside the second FOUP at the first flow rate;
determining, by a third sensor disposed at the outlet of the second FOUP, a fourth RH at the outlet of the second FOUP;
determining a relationship between the third RH and the fourth RH; and
updating the model based on the relationship between the third RH and the fourth RH.

16. The system of claim 15, wherein the model comprises a trained machine learning model.

17. The system of claim 13, wherein the controller is further configured to:
determine the second RH inside the first FOUP is greater than a threshold value; and
transmit a message to a load port unit (LPU) of a processing chamber comprising the first FOUP.

18. The system of claim 13, wherein the first sensor comprises a micro electromechanical systems (MEMS) sensor.

19. The system of claim 15, wherein the second sensor is disposed on one or more slots of the second FOUP.

20. A method comprising:
supplying a fluid, at a first flow rate, through an inlet of a substrate carrier;
at least partially purging the fluid through an outlet of the substrate carrier for a first period of time;
determining, by a first sensor disposed inside the substrate carrier, a value of a first property inside the substrate carrier;
determining, by a second sensor disposed at the outlet of the substrate carrier, a second value of the first property at the outlet of the substrate carrier;
determining a relationship between the first value of the first property and the second value of the first property; and
updating a model based on the relationship between the first value of the first property and the second value of the first property, wherein the model is trained to process a measured value of the first property at an outlet of a new substrate carrier to determine an estimated value of the first property within the new substrate carrier.
